(19) United States
(12) Patent Application Publication
(10) Pub. No.: US 2011/0086070 A1
(43) Pub. Date: Apr. 14, 2011

(54) Orally disintegrating compositions of rhein or diacerein

(76) Inventors: Munish Talwar, Panchkula (IN); Rahul Dabre, Nagpur (IN); Ritesh Kapoor, Mandi (IN); Himanshu Verma, Ranchi (IN); Nitin Jain, Gwalior (IN); Girish Kumar Jain, Delhi (IN)

(21) Appl. No.: 12/934,122

(22) PCT Filed: Sep. 26, 2008
(86) PCT No.: PCT/IB08/53925

§ 371 Date: Dec. 26, 2010

(30) Foreign Application Priority Data
Mar. 24, 2008 (IN) 597/MUM/2008
Mar. 24, 2008 (IN) 598/MUM/2008

(51) Int. Cl. A61K 31/235; A61K 31/192; A61K 9/00

(52) U.S. Cl. 424/400; 514/510; 514/569

(57) ABSTRACT

The invention relates to orally disintegrating pharmaceutical compositions comprising rhein or diacerein, or salts or esters or prodrugs thereof, and processes for preparing such compositions.

### FIELD OF THE INVENTION

The invention relates to orally disintegrating pharmaceutical compositions comprising rhein or diacerein, or salts or esters or prodrugs thereof, and processes for preparing such compositions.

### BACKGROUND OF THE INVENTION

Chemically, rhein is 9,10-dihydro-4,5-dihydroxy-9,10-dioxo-2-anthracene carboxylic acid having a structure of Formula I and diacerein is 4,5-bis(acetyloxy)9,10-dihydro-4,5-dihydroxy-9,10-dioxo-2-anthracenecarboxylic acid having a structure of Formula II. Diacerein is widely used in the treatment of osteoarthritis and has a unique mode of action that differentiates it from non-steroidal anti-inflammatory drugs (NSAIDs) and other conventional forms of drug therapy.

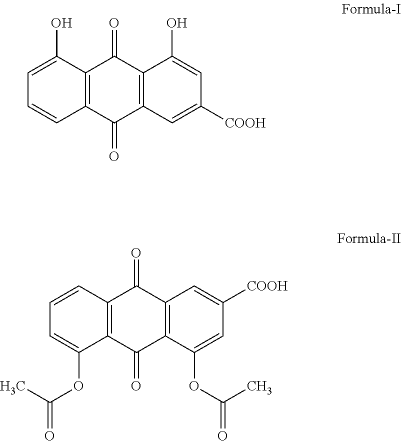

Diacerein is practically insoluble in solvents such as water, alcohols, acetone, dichloromethane and chloroform, which are generally used in pharmaceutical preparations. Although diacerein can be administered by oral route but it cannot be completely absorbed by the digestive tract, and this incomplete absorption may result in undesirable side effects such as, soft stools.

In order to overcome these problems, various derivatives, pharmaceutical compositions and specific galenic forms have been proposed in the literature. For example, European patent, EP 243,968 discloses a potassium salt of diacerein, which is water-soluble and can be used in the preparation of compositions for parenteral administration.

European Patent No EP904060 discloses pharmaceutical compositions of rhein or diacerein, wherein rhein or diacerein is co-micronized with sodium lauryl sulfate.

European Patent Nos. EP263083; 264989 and 446753 disclose controlled release or delayed release compositions like multiplicity of pellets coated with drug and coating membrane or granules of drug coated with polymers or loading polymeric particles of water swellable cross-linked polymer with drug.

U.S. Pat. Nos. 5,225,192 and 5,569,469 describe different poorly soluble medicaments supported on polymeric particles of water swellable cross-linked polymer with drug.

U.S. Pat. No. 5,952,383 and European Patent No. EP 862423 provide pharmaceutical compositions of diacerein, rhein and their salts along with liquid support systems like oils, suspending agents, homogenizing agents and other excipients.

Currently, diacerein is marketed as capsules in 50 mg strength under the trade name Art 50® and given twice a day. The capsule forms are, however, difficult to swallow, especially for geriatric patients. Further, the fear of swallowing, or choking on such solid shaped articles is still a concern in certain populations especially, geriatrics. It is estimated that almost 50% of the population have problems of swallowing tablets or capsules (Seager in Journal of Pharmacol. and Pharm. Pages 375-382, 1998). The capsule dosage forms become sticky when wetted by saliva, and if a patient experiences difficulty in swallowing on its first attempt, then the capsule must often be discarded. Furthermore, if a capsule partially dissolves in a patient's mouth, as can result from unsuccessful swallowing or the capsule getting stuck in the orthodontic appliance, the resulting very unpleasant taste can make it difficult to persuade the patient to take another dose. Additionally, these dosage forms are difficult to carry, store and handle. All these difficulties associated with capsules result in decreased patient compliance.

Orally dissolving formulations of rhein or diacerein are beneficial for many reasons and can be administered without liquid, anywhere, anytime. The use of these formulations is particularly advantageous in situations where patients have difficulty in swallowing especially, geriatrics and those with neurological disorders. In addition to convenient dosing and better patient compliance, the formulation disintegrates very fast and thus results in a rapid release. Design of orally disintegrating tablet requires a significant amount of research work in order to develop a composition that maintains enough porosity inside the compressed tablet for fast dissolving or fast melting while maintaining the mechanical strength of the tablet. It was a significant challenge to obtain suitable orally disintegrating tablets of rhein or diacerein, which have high bioavailability, palatable to the patient and are stable. An ideal orally disintegrating tablet should also have good organoleptic properties, such as an immediate disintegration of tablet to prevent any adverse feeling in the mouth, and sufficient mechanical strength to allow for appropriate packaging operations. The inventors have overcome these challenges and were able to develop orally disintegrating tablets comprising rhein or diacerein.

Although the concept of oral films or strips comprising certain pharmaceutically active substances is known in the art as early as 1974, for example through U.S. Pat. No. 4,136,162, but such oral dosage forms have hardly been commercialized during the last 30 years. There are no prescription products in the market based on oral thin film or strips till date. This may be due to a lot of technical and other obstacles preventing commercialization, despite known benefits of oral films or strips. The inventors were also able to develop oral films or strips comprising rhein or diacerein.

### SUMMARY OF THE INVENTION

In one general aspect there is provided an orally disintegrating pharmaceutical composition comprising rhein or diacerein, or salts or esters or prodrugs thereof, and one or more pharmaceutically acceptable excipients.

Embodiments of the pharmaceutical composition may include one or more of the following features. For example, the composition may be in the form of a tablet, granules, particles or pellets.

In another general aspect there is provided an orally disintegrating pharmaceutical composition comprising rhein or diacerein, or salts or esters or prodrugs thereof, wherein the composition exhibits a dissolution profile such that more than 70% of rhein or diacerein or salts or esters or prodrugs thereof is released within 15 minutes, wherein the release rate is measured in Apparatus 2 (USP, Dissolution, paddle, 75 rpm) using 1000 ml of pH 5.7 phosphate buffer at 37° C.±0.5° C.

In another general aspect there is provided an orally dissolving pharmaceutical composition in the form of a film or strips, comprising rhein or diacerein, or salts or esters or prodrugs thereof, and one or more pharmaceutically acceptable film-forming polymers.

In another general aspect there is provided an orally dissolving pharmaceutical composition in the form of a film or strips, comprising rhein or diacerein, or salts or esters or prodrugs thereof, and one or more pharmaceutically acceptable excipients, wherein the composition exhibits a dissolution profile such that more than 80% of rhein or diacerein or salts or esters or prodrugs thereof is released within 30 minutes, wherein the release rate is measured in Apparatus 2 (USP, Dissolution, paddle, 75 rpm) using 1000 ml of pH 5.7 phosphate buffer at 37° C.±0.5° C.

In another general aspect there is provided a process for the preparation of a pharmaceutical composition in the form of a film or strips. The process includes dissolving or dispersing one or more film-forming polymers in an aqueous medium; mixing rhein or diacerein or salts or esters or prodrugs thereof; and casting the mixture into films of a suitable size.

Embodiments of the pharmaceutical composition may include one or more of the following features. The pharmaceutical composition may further include one or more pharmaceutically acceptable excipients. For example, the pharmaceutically acceptable excipients may include one or more of fillers, binders, lubricants, sweeteners, coloring and flavoring agents, glidants, disintegrants, and the like.

The details of one or more embodiments of the inventions are set forth in the description below. Other features, objects and advantages of the inventions will be apparent from the description and claims.

### DETAILED DESCRIPTION OF THE INVENTION

The inventors have developed orally disintegrating compositions in the form of tablets, thin films or strips, which have reproducible disintegration time, good chemical stability and organoleptic properties.

The pharmaceutical compositions of the invention may be prepared by physical mixing, wet mixing, spray congealing, hot melt, anti-solvent, microfluidization, spray drying and freeze drying.

According to one embodiment, the pharmaceutical composition of the invention may be prepared by mixing rhein or diacerein or salts or esters or prodrugs thereof with other pharmaceutically acceptable excipients, and optionally compressing the mixture into tablets.

According to another embodiment, the pharmaceutical composition of the invention may be prepared by suspending a mixture of rhein or diacerein or salts or esters or prodrugs thereof with other pharmaceutically acceptable excipients in a surfactant solution and granulating the mixture of other pharmaceutically acceptable excipients with the suspension, drying the granules, and optionally compressing the granules into tablets.

The tablets of the invention may disintegrate in the oral cavity of a human in less than 45 seconds, for example in less than 30 seconds.

The tablet may vary in shapes such as oval, round, triangle, almond, peanut, pentagonal, trapezoidal, parallelogram, and the like.

The pH of a 0.5-1% w/w dispersion of orally disintegrating tablet of the invention may be in the range of 4.0-7.0.

The pharmaceutical composition of the invention may include one or more of pharmaceutically acceptable excipients selected from one or more of binders, fillers, disintegrants, glidants, lubricants, surfactants, sweeteners and flavors.

Suitable binders may include one or more of povidone, starch, stearic acid, gums, celluloses, alginic acids, chitosan, chitin, polyethylene glycol, and the like.

Suitable fillers may include one or more of saccharose, glucose, fructose, maltose, maltitol, mannitol, dextrins such as maltodextrins; xylitol, sorbitol, microcrystalline cellulose, titanium dioxide, calcium phosphate, calcium sulfate, kaolin, dry starch, powdered sugar, silicates such as magnesium aluminium silicate, and the like.

Suitable disintegrants may include one or more of starch, croscarmellose sodium, crospovidone, sodium starch glycolate, and the like.

Suitable glidants may include one or more of colloidal silicon dioxide, talc or cornstarch, and the like.

Suitable lubricants may include one or more of magnesium stearate, zinc stearate, calcium stearate, stearic acid, sodium stearyl fumarate, hydrogenated vegetable oil, glyceryl behenate, and the like.

Suitable surfactants may include one or more of amphoteric, non-ionic, cationic or anionic surfactants. For example, sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer, cremophore RH 40, and the like.

Suitable sweeteners may include one or more of monosaccharides, disaccharides and polysaccharides, e.g. xylose, ribose, glucose, mannose, galactose, fructose, sucrose, maltose, invert sugar, partially hydrolyzed starch, corn syrup solids, mannitol, xylitol, D-sorbitol, erythritol, pentitol, hexitol, malitol, dihydrochalcones, monellin, steviosides or glycyrrhizin; saccharin in free acid form, soluble saccharin salts, e.g. sodium or calcium saccharin salts, cyclamate salts or acesulfame K; dipeptide based sweeteners, such as L-aspartic acid derived sweeteners, e.g. aspartame; water-soluble sweeteners derived from naturally occurring water-soluble sweeteners, e.g. sucralose; and protein based sweeteners, e.g. thaumatococcus danielli (Thaumatin I and II), and the like.

Suitable flavoring agents may include one or more of natural, “natural-like” and artificial flavors. The flavoring agents may be selected from synthetic flavor oils, flavoring aromatics, oleo-resins and extracts derived from plants, leaves, flowers or fruits.

Representative flavors may include one or more of spearmint oil, cinnamon oil, peppermint oil, clove oil, bay oil, thyme oil, cedar leaf oil, oil of nutmeg, oil of sage, oil of bitter almonds, vanilla, chocolate, coffee, cocoa and citrus oil, lemon, orange, cherry, grape, lime or grapefruit, and fruit essences, e.g. apple, pear, peach, strawberry, raspberry, cherry, plum, pineapple or apricot; mints such as peppermint (including menthol, especially levomenthol), aldehydes and esters, e.g. cinnamyl acetate, cinnamaldehyde, citral, diethylacetal, dihydrocarvyl acetate, eugenyl formate or p-methylanisol; alpha-citral (geranial) and beta-citral (neral); decanal; ethyl vanillin; piperonal (heliotropine); vanillin; alpha-amyl cinnamaldehyde; butyraldehyde; valeraldehyde; citronellal; decanal; aldehyde C-8; aldehyde C-9; aldehyde C-12; 2-ethyl butyraldehyde; hexenal, i.e. trans-2; tolyl aldehyde; veratraldehyde; 2,6-dimethyl-5-heptenal(melonal); 2-6-dimethyloctanal; 2-dodecenal, and the like.

The tablets of the invention may optionally include usual auxiliaries known in the art such as saliva stimulating agents like citric acid, lactic acid, malic acid, succinic acid, ascorbic acid, adipic acid, fumaric acid, tartaric acids; cooling sensation agents like maltitol, monomenthyl succinate, ultracool; stabilizers like gums, agar; taste masking agents like acrylic polymers, copolymers of acrylates, celluloses, resins; coloring agents like titanium dioxide, natural food colors, dyes suitable for food, drug and cosmetic applications; preservatives like alpha-tocopherol, citric acid, butylated hydroxytoluene, butylated hydroxyanisole, ascorbic acid, fumaric acid, malic acid, sodium ascorbate or ascorbic acid palmitate or effervescing agents like citric acid, tartaric acid, sodium bicarbonate, sodium carbonate, and the like.

According to another embodiment, the pharmaceutical film composition of the invention may be prepared by dissolving film-forming polymers and other pharmaceutically acceptable excipients in an aqueous medium; mixing rhein or diacerein or salts or esters or prodrugs thereof; and finally casting the mixture into films or strips of a suitable size.

The film or strips of the invention may disintegrate in the oral cavity of a human in less than 45 seconds, for example in less than 30 seconds.

The films or strips of the invention may have a thickness of 1 mm or less, for example 0.5 mm or less. Further, the films of the invention may not be limited to any particular size and may be rectangular, square, or round. The film or strips of the invention may be mucoadhesive in nature.

The rhein or diacerein or salts or esters or prodrugs thereof may be present in the film either in dissolved or uniformly dispersed state.

The films or strips of the invention may be prepared by hot-melt extrusion, solid dispersion extrusion, rolling, semi-solid casting and solvent coating.

The pharmaceutically acceptable polymers may include one or more of film forming polymers such as methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phtalate, cellulose acetate butyrate, amylose, dextran, casein, pullulan, gelatine, pectin, chitin, chitosan, elsiman, zein, gluten, soy protein isolate, whey protein isolate, agar, carrageenan, xanthan gum, tragacanth, guar gum, acacia gum, arabic gum, polyethylene glycol, polyethylene oxide, polyvinyl pyrrolidone, polyvinyl alcohol, cyclodextrin, carboxyvinyl polymers, polycarbophils, sodium alginate, polyacrylic acid, methylmethacrylate, and the like.

The film or strips of the invention may include one or more of pharmaceutically acceptable excipients. For example, the excipients may include one or more of plasticizers, emulsifiers, sweeteners, and flavors.

Suitable plasticizers may include one or more of polyethylene glycol, propylene glycol, glycerin, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol, and the like.

Suitable emulsifiers may include one or more of polyoxyethylene glycerol esters of fatty acids, such as Tagats; polooxylated castor oil, ethylene glycol esters, such as glycol stearate and distearate; propylene glycol esters, such as propylene glycol myristate; glyceryl esters of fatty acids, such as glyceryl stearates and monostearates; sorbitan esters, such as spans and tweens; polyglyceryl esters, such as polyglyceryl 4-oleate; fatty alcohol ethoxylates, such as Brij type emulsifiers; ethoxylated propoxylated block copolymers, such as poloxamers; polyethylene glycol esters of fatty acids, such as Labrafils, Labrafacs, and Labrasols; cremophores; glycerol monocaprylate/caprate, such as Campmul CM 10; Gelucire, Capryol, Captex, Acconon, transcutol, triacetin, vitamin E TPGS, and the like.

Suitable sweeteners may include one or more of monosaccharides, disaccharides and polysaccharides, e.g. xylose, ribose, glucose, mannose, galactose, fructose, sucrose, maltose, invert sugar, partially hydrolyzed starch, corn syrup solids, mannitol xylitol, D-sorbitol, erythritol, pentitol, hexitol, malitol, dihydrochalcones, monellin, steviosides or glycyrrhizin; saccharin in free acid form, soluble saccharin salts, e.g. sodium or calcium saccharin salts, cyclamate salts or acesulfame K; dipeptide based sweeteners, such as L-aspartic acid derived sweeteners, e.g. aspartame; water-soluble sweeteners derived from naturally occurring water-soluble sweeteners, e.g. sucralose; and protein based sweeteners, e.g. thaumatococcus danielli (Thaumatin I and II), and the like.

Suitable flavoring agents may include natural, “natural-like” and artificial flavors. These flavors may be selected from synthetic flavor oils, flavoring aromatics, oleo-resins and extracts derived e.g. from plants, leaves, flowers or fruits.

Representative flavors may include one or more of spearmint oil, cinnamon oil, peppermint oil, clove oil, bay oil, thyme oil, cedar leaf oil, oil of nutmeg, oil of sage, oil of bitter almonds, vanilla, chocolate, coffee, cocoa and citrus oil, lemon, orange, cherry, grape, lime or grapefruit, and fruit essences, e.g. apple, pear, peach, strawberry, raspberry, cherry, plum, pineapple or apricot; mints such as peppermint (including menthol, especially levomenthol), aldehydes and esters, e.g. cinnamyl acetate, cinnamaldehyde, citral, diethylacetal, dihydrocarvyl acetate, eugenyl formate or p-methylanisol; alpha-citral (geranial) and beta-citral (neral); decanal; ethyl vanillin; piperonal (heliotropine); vanillin; alpha-amyl cinnamaldehyde; butyraldehyde; valeraldehyde; citronellal; decanal; aldehyde C-8; aldehyde C-9; aldehyde C-12; 2-ethyl butyraldehyde; hexenal, i.e. trans-2; tolyl aldehyde; veratraldehyde; 2,6-dimethyl-5-heptenal (melonal); 2-6-dimethyloctanal; 2-dodecenal, and the like.

The films of the invention may optionally include usual auxiliaries known in the art such as saliva stimulating agents like citric acid, lactic acid, malic acid, succinic acid, ascorbic acid, adipic acid, fumaric acid, tartaric acids; cooling sensation agents like maltitol, monomenthyl succinate, ultracool; fillers like saccharose, glucose, fructose, maltose, maltitol, mannitol, dextrins such as maltodextrins; xylitol, sorbitol, microcrystalline cellulose, titanium dioxide; stabilizers like gums, agar; coloring agents like titanium dioxide, natural food colors, dyes suitable for food, drug and cosmetic applications; disintegration agents like croscarmellose sodium; preservatives like alpha-tocopherol, citric acid, butylated hydroxytoluene, butylated hydroxyanisole, ascorbic acid, fumaric acid, malic acid, sodium ascorbate or ascorbic acid palmitate or pH regulators like sodium bicarbonate, sodium carbonate; antitacking agents like talc, magnesium stearate, sodium stearyl fumarate, stearic acid, and the like.

The invention is further illustrated by the following examples which are provided merely to be exemplary of the invention and do not limit the scope of the invention. Certain modifications and equivalents will be apparent to those skilled in the art and are intended to be included within the scope of the invention.

### EXAMPLE 1

TABLE 1

| S.N. | Ingredients | % w/w |
|---|---|---|
| 1 | Diacerein | 7.5-37.50 |
| 2 | Microcrystalline Cellulose | 5-80 |
| 3 | Crospovidone | 2-10 |
| 4 | Croscarmellose Sodium | 2-10 |
| 5 | Hydroxy propyl Cellulose | 2-10 |
| 6 | Aspartame | 0.1-5 |
| 7 | Acesulfame | 0.1-5 |
| 8 | Butterscotch and Tutti-frutti flavor | 0.1-5 |
| 9 | Mannitol | 5-80 |
| 10 | Citric Acid | 0.25-10 |
| 11 | Sodium Bi Carbonate | 0.25-10 |
| 12 | Colloidal Silicon Dioxide | 0.5-2.5 |
| 13 | Magnesium Stearate | 0.5-2 |

Procedure: Butterscotch and tutti-frutti flavor, aspartame, acesulfame were triturated together and added to microcrystalline cellulose by sifting to form a blend. The blend was admixed with diacerein, mannitol, polyplasdone, croscarmellose sodium, citric acid, sodium bicarbonate and hydroxypropyl cellulose to form a uniform mixture. The mixture was blended with colloidal silicon dioxide and lubricated with magnesium stearate. The lubricated blend was compressed into suitable sized tablets using a suitable tooling. The tablet disintegrated in about 20-27 seconds.

### EXAMPLE 2

TABLE 2

| S.N. | Ingredients | % w/w |
|---|---|---|
| 1 | Diacerein | 7.5-37.50 |
| 2 | Methacrylic acid divinyl benzene copolymer | 1-10 |
| | potassium salt | |
| 3 | Microcrystalline Cellulose | 5-75 |
| 4 | Crospovidone | 2-10 |
| 5 | Croscarmellose Sodium | 2-10 |
| 6 | Hydroxy propyl Cellulose | 2-10 |
| 7 | Aspartame | 0.1-5 |
| 8 | Pineapple flavor | 0.1-5 |
| 9 | Mannitol | 5-75 |
| 10 | Colloidal Silicon Dioxide | 0.25-2.5 |
| 11 | Magnesium Stearate | 0.25-2 |

Procedure: Pineapple flavor and aspartame were added to microcrystalline cellulose by sifting to form a blend. Diacerein and methacrylic acid divinyl benzene copolymer potassium salt were sifted together and added to the above blend. The blend was admixed with mannitol, polyplasdone, croscarmellose sodium, and hydroxypropyl cellulose to form a uniform mixture. The mixture was blended with colloidal silicon dioxide. The blend was lubricated with magnesium stearate. The lubricated blend was compressed into suitable sized tablets using a suitable tooling. The tablet disintegrated in about 30 seconds.

### EXAMPLE 3

TABLE 3

| S.N. | Ingredients | % w/w |
|---|---|---|
| 1 | Diacerein | 7.5-37.50 |
| 2 | Magnesium aluminum Silicate | 2-20 |
| 3 | Microcrystalline Cellulose | 5-80 |
| 4 | Crospovidone | 2-10 |
| 5 | Croscarmellose Sodium | 2-10 |
| 6 | Hydroxy propyl Cellulose | 2-10 |
| 7 | Aspartame | 0.1-5 |
| 8 | Lemon flavor | 0.1-5 |
| 9 | Mannitol | 5-80 |
| 10 | Colloidal Silicon Dioxide | 0.25-2.5 |
| 11 | Magnesium Stearate | 0.25-2 |

Procedure: Lemon flavor, magnesium aluminum silicate and aspartame were added to microcrystalline cellulose by sifting to form a blend. The blend was admixed with diacerein, mannitol, polyplasdone, croscarmellose sodium, and hydroxypropyl cellulose to form a uniform mixture. The mixture was blended with colloidal silicon dioxide. The blend was lubricated with magnesium stearate. The lubricated blend was compressed into suitable sized tablets using a suitable tooling. The tablet disintegrated in about 20-27 seconds.

### EXAMPLE 4

TABLE 4

| S.N. | Ingredients | % w/w |
|---|---|---|
| 1 | Diacerein | 7.5-37.50 |
| 2 | Microcrystalline Cellulose | 5-80 |
| 3 | Crospovidone | 2-10 |
| 4 | Croscarmellose Sodium | 2-10 |
| 5 | Hydroxy propyl Cellulose | 2-10 |
| 6 | Sodium Lauryl Sulfate | 0.5-2.5 |
| 7 | Docusate Sodium | 0.1-2.5 |
| 8 | Purified Water | q.s. |
| 9 | Isopropyl Alcohol | q.s. |
| 10 | Aspartame | 0.1-5 |
| 11 | Acesulfame | 0.1-5 |
| 12 | Butterscotch and tutty fruity flavor | 0.1-5 |
| 13 | Mannitol | 5-80 |
| 14 | Glycine | 0.1-5 |
| 15 | Colloidal Silicon Dioxide | 0.25-2.5 |
| 16 | Magnesium Stearate | 0.25-2 |

Procedure: Docusate Sodium was dissolved in isopropyl alcohol and sodium lauryl sulphate was dissolved in purified water and the two solutions were mixed together. Diacerein, acesulfame, aspartame, glycine, and flavor were sifted together and subjected to homogenization with the above solution. The suspension was used for granulation using base of mannitol. The granulated mass was dried and subsequently sized to form uniform granules. Microcrystalline Cellulose, polyplasdone, croscarmellose sodium, and hydroxypropyl cellulose were admixed along with granules and blended with colloidal silicon dioxide. The blend was lubricated with magnesium stearate. The lubricated blend was compressed into suitable sized tablets using a suitable tooling.

### EXAMPLE 5

TABLE 5

| S.N. | Ingredients | % w/w |
|---|---|---|
| 1 | Diacerein | 7.5-37.50 |
| 2 | Pregelatinized Starch | 5-20 |
| 3 | Crospovidone | 2-10 |
| 4 | Croscarmellose Sodium | 2-10 |
| 5 | Hydroxy propyl Cellulose | 2-10 |
| 6 | Poloxamer | 2-10 |
| 7 | Acesulfame | 0.1-5 |
| 8 | Sucralose | 0.1-5 |
| 9 | Tropical banana flavor | 0.1-5 |
| 10 | Lactose | 5-80 |
| 11 | Mannitol | 5-80 |
| 12 | Menthol | 0.01-0.5 |
| 13 | Colloidal Silicon Dioxide | 0.25-2.5 |
| 14 | Sodium Stearyl fumarate | 0.25-2 |

Procedure: Poloxamer was dissolved in purified water. Diacerein, acesulfame, sucralose and flavors were sifted together and subjected to homogenization with the above solution. The suspension was used for granulation using base of lactose. The granulated mass was dried. Menthol was dissolved in isopropyl alcohol and then sprayed/added on to the lactose drug loaded base granules and dried. The dried granules were sized to form uniform granules. Pregelatinized starch, mannitol, polyplasdone, croscarmellose sodium, and hydroxypropyl cellulose were admixed along with granules and blended with colloidal silicon dioxide. The blend was lubricated with magnesium stearate. The lubricated blend was compressed into suitable sized tablets using a suitable tooling.

### EXAMPLE 6

TABLE 6

| S.N. | Ingredients | % w/w |
|---|---|---|
| 1 | Diacerein | 5-20 |
| 2 | Pullulan | 5-15 |
| 3 | HPMC | 5-15 |
| 4 | Luster clear | 5-15 |
| 5 | Carrageenan | 0.1-5 |
| 6 | Xanthan Gum | 0.1-5 |
| 7 | Locust Bean Gum | 0.1-5 |
| 8 | Maltodextrin | 5-15 |
| 9 | Xylitol | 5-15 |
| 10 | Propylene Glycol | 0.5-5 |
| 11 | Glycerin | 0.5-5 |
| 12 | Tween 80 | 0.5-5 |
| 13 | Olive Oil | 0.5-5 |
| 14 | Sucralose | 5-10 |
| 15 | Acesulfame potassium | 0.5-5 |
| 16 | Peppermint flavor | 10-25 |
| 17 | Menthol | 0.5-5 |
| 18 | Orange-vanilla flavor | 0.5-5 |
| 19 | Water | q.s. |

Procedure: Pullulan, HPMC, lusterclear, carrageenan, locust bean gum & xanthan gum were added to purified water with continuous stirring and allowed to dissolve completely. Sucralose, xylitol, acesulfame potassium, maltodextrin, orange vanilla flavor & peppermint flavor were added to the above mixture and stirred for few minutes. Tween-80, propylene glycol, glycerin, olive oil and menthol were mixed and added to the above mixture and stirred for few minutes. Diacerein was added to the above mixture and stirred for few minutes. The final mixture thus obtained was casted into films of suitable size.

### EXAMPLE 7

TABLE 7

| S.N. | Ingredients | (% w/w) |
|---|---|---|
| 1 | Diacerein | 5-20 |
| 2 | Xanthan gum | 0.1-5 |
| 3 | Locust bean gum | 0.1-5 |
| 4 | Carrageenan | 0.1-5 |
| 5 | HPMC | 5-15 |
| 6 | CMC Sodium | 1-5 |
| 7 | Pro sweet | 1-5 |
| 8 | Sucralose | 5-10 |
| 9 | Mannitol | 5-15 |
| 10 | Water | q.s. |
| 11 | Maltitol | 5-15 |
| 12 | Menthol | 0.5-5 |
| 13 | Mixed Fruit Flavour | 0.5-5 |
| 14 | Polysorbate 80 | 0.5-5 |
| 15 | Vitamin E TPGS | 0.5-5 |
| 16 | Glycerin | 0.5-5 |
| 17 | Olive oil | 0.5-5 |

Procedure: Vitamin E TPGS was added to water having temperature 40-50° C. and stirred for few minutes. ProSweet, sucralose, xanthan gum, locust bean gum and carrageenan, HPMC, CMC Sodium were added to TPGS solution under continuous stirring. The mixture was stirred for few minutes. Diacerein was added to the above mixture and the above mixture was allowed to stir for few minutes (Mixture A). Glycerin was heated to about 40-50° C. and menthol was added to it and allowed to dissolve by stirring (Mixture B). Olive oil, mixed fruit flavour and polysorbate 80 were added to the mixture B to form mixture C. The mixture C was added to the mixture A under continuous stirring (Mixture D). The mixture D was casted into films of suitable size.

### EXAMPLE 8

TABLE 8

| S.N. | Ingredients | (% w/w) |
|---|---|---|
| 1 | Diacerein | 5-20 |
| 2 | HPMC | 5-15 |
| 3 | Sucralose | 5-10 |
| 4 | Water | q.s. |
| 5 | Ethanol | q.s. |
| 6 | Menthol | 0.5-5 |
| 7 | Mixed Fruit Flavour | 0.5-5 |
| 8 | Peppermint Flavour | 0.5-5 |
| 9 | PEG 400 | 0.5-5 |
| 10 | Vitamin E TPGS | 0.5-5 |
| 11 | Glycerin | 0.5-5 |

Procedure: Vitamin E TPGS was added to water having temperature 40-50° C. under continuous stirring. The mixture was stirred for few minutes (Mixture A). Sucralose, peppermint flavour and HPMC were added to ethanol under continuous stirring. The mixture was stirred for few minutes (Mixture B). The mixture B was added to the mixture A under continuous stirring (Mixture C). Diacerein was added to the mixture C and allowed to stir for few minutes (Mixture D). Glycerin was heated to about 40-50° C. and menthol crystals were added to it and allowed to dissolve by stirring (Mixture E). Mixed fruit flavour and PEG 400 were added to the mixture E (Mixture F). The mixture F was added to the mixture B under continuous stirring and stirred for few minutes (Mixture G). The mixture G was casted into films of suitable size.

While the invention has been described in terms of its specific embodiments, certain modifications and equivalents will be apparent to those skilled in the art and are intended to be included within the scope of the invention.

1. An orally disintegrating pharmaceutical composition comprising rhein or diacerein, or salts or esters or prodrugs thereof, and one or more pharmaceutically acceptable excipients.

2. The composition of claim 1, wherein the composition disintegrates in the oral cavity of a human in less than about 45 seconds.

3. The composition of claim 1, wherein the pharmaceutically acceptable excipients comprise one or more of binders, fillers, disintegrants, glidants, lubricants, surfactants, sweeteners and flavors.

4. The composition of claim 3, wherein the binder comprises one or more of, povidone, starch, stearic acid, gums, celluloses, alginic acids, chitosan, chitin, and polyethylene glycol.

5. The composition of claim 3, wherein the filler comprises one or more of saccharose, glucose, fructose, maltose, maltitol, mannitol, dextrins such as maltodextrins; xylitol, sorbitol, microcrystalline cellulose, titanium dioxide, calcium phosphate, calcium sulfate, kaolin, dry starch, powdered sugar and silicates.

6. The composition of claim 3, wherein the disintegrant comprises one or more of starch, croscarmellose sodium, crospovidone and sodium starch glycolate.

7. The composition of claim 3, wherein the surfactant comprises one or more of sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer and cremophore RH 40.

8. The pharmaceutical composition of claim 1, wherein the composition is in the form of a tablet, granules, particles or pellets.

9. The pharmaceutical composition of claim 1, wherein the composition exhibits a dissolution profile such that more than 70% of rhein or diacerein or salts or esters or prodrugs thereof is released within 15 minutes, wherein the release rate is measured in Apparatus 2 (USP, Dissolution, paddle, 75 rpm) using 1000 ml of pH 5.7 phosphate buffer at 37° C.±0.5° C.

10. An orally dissolving pharmaceutical composition in the form of a film or strips comprising rhein or diacerein, or salts or esters or prodrugs thereof, and one or more pharmaceutically acceptable film-forming polymers.

11. The composition of claim 10, wherein the pharmaceutically acceptable film forming polymer comprises one or more of methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phtalate, cellulose acetate butyrate, amylose, dextran, casein, pullulan, gelatine, pectin, chitin, chitosan, elsiman, zein, gluten, soy protein isolate, whey protein isolate, agar, carrageenan, xanthan gum, tragacanth, guar gum, acacia gum, arabic gum, polyethylene glycol, polyethylene oxide, polyvinyl pyrrolidone, polyvinyl alcohol, cyclodextrin, carboxyvinyl polymers, sodium alginate, polyacrylic acid, and methylmethacrylate.

12. The composition of claim 10, wherein the composition disintegrates in the oral cavity of a human in less than about 45 seconds.

13. The composition of claim 10, wherein the composition has a thickness of 1 mm or less.

14. The composition of claim 10, wherein the composition further comprises one or more pharmaceutically acceptable excipients comprising plasticizers, emulsifiers, sweeteners, and flavors.

15. The composition of claim 14, wherein the plasticizer comprises one or more of polyethylene glycol, propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, and sorbitol.

16. The composition of claim 14, wherein the emulsifier comprises one or more of polyoxyethylene glycerol esters of fatty acids, polooxylated castor oil, ethylene glycol esters, propylene glycol esters, glyceryl esters of fatty acids, sorbitan esters, polyglyceryl esters, fatty alcohol ethoxylates, ethoxylated propoxylated block copolymers, polyethylene glycol esters of fatty acids, glycerol monocaprylate/caprate, Gelucire, Capryol, Captex, Acconon, transcutol, vitamin E TPGS and triacetin.

17. The composition of claim 10, wherein the composition exhibits a dissolution profile such that more than 80% of rhein or diacerein or salts or esters or prodrug thereof is released within 30 minutes, wherein the release rate is measured in Apparatus 2 (USP, Dissolution, paddle, 75 rpm) using 1000 ml of pH 5.7 phosphate buffer at 37° C.±0.5° C.

18. A process for the preparation of a pharmaceutical composition in the form of a film or strips comprising rhein or diacerein, or salts or esters or prodrugs thereof, the process comprising dissolving or dispersing film-forming polymers in an aqueous medium; mixing rhein or diacerein or salts or esters or prodrugs thereof; and casting the mixture into films or strips of a suitable size.

19. The process of claim 18, wherein the film forming polymer comprises one or more of methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phtalate, cellulose acetate butyrate, amylose, dextran, casein, pullulan, gelatine, pectin, chitin, chitosan, elsiman, zein, gluten, soy protein isolate, whey protein isolate, agar, carrageenan, xanthan gum, tragacanth, guar gum, acacia gum, arabic gum, polyethylene glycol, polyethylene oxide, polyvinyl pyrrolidone, polyvinyl alcohol, cyclodextrin, carboxyvinyl polymers, sodium alginate, polyacrylic acid, and methylmethacrylate.

20. The process of claim 18, wherein the composition further comprises one or more other pharmaceutically acceptable excipients comprising plasticizers, emulsifiers, sweeteners, and flavors.
